(19) United States
(12) Patent Application Publication
(10) Pub. No.: US 2008/0195140 A1
(43) Pub. Date: Aug. 14, 2008

(54) Delivery system for an embolic protection device

(75) Inventors: Subbarao Myla, Newport Beach, CA (US); Jason C. Urbanski, Ellettsville, IN (US); Sarah E. Reeves, Cory, IN (US)

Correspondence Address: COOK GROUP PATENT OFFICE, P.O. BOX 2269, BLOOMINGTON, IN, 47402, US

(73) Assignee: Cook Incorporated, Bloomington, IN (US)

(21) Appl. No.: 11/999,812

(22) Filed: Dec. 7, 2007

Related U.S. Application Data
(60) Provisional application No. 60/873,902, filed on Dec. 8, 2006.

(51) Int. Cl. A61M 29/00

(52) U.S. Cl. 606/200

(57) ABSTRACT

A delivery system for an embolic protection device is described herein. The delivery system may allow the embolic protection device to be transported and deployed within vessels having a tortuous anatomy. The delivery system includes a delivery catheter having a bend near a distal end thereof. The bend has a non-included angle of greater than about 40 degrees relative to a longitudinal axis of the catheter. The delivery system further comprises an embolic protection device disposed within the delivery catheter.

### RELATED APPLICATIONS

The present patent document claims the benefit of the filing date under 35 U.S.C. §119(e) of Provisional U.S. Patent Application Ser. No. 60/873,902, filed Dec. 8, 2006, which is hereby incorporated by reference.

### TECHNICAL FIELD

The present disclosure relates to a delivery system for intraluminal medical devices, in particular, to a delivery system for embolic protection devices.

### BACKGROUND

Balloon angioplasty and stenting procedures are widely used in the treatment of stenoses of the coronary arteries as an alternative to invasive bypass surgeries. However, the inflation of a balloon or placement of a stent at the stenosed region can dislodge embolic particles from the lesion that may travel downstream (distal) of the stenosis. In certain critical arteries, such as carotid arteries, the embolic particles may become trapped in small-diameter blood vessels of the brain and may cause a stroke.

To increase the safety of carotid angioplasty and stenting procedures, embolic protection devices have been developed as a means to capture embolic particles that have been dislodged from a stenosis. Such devices may be deployed within a vessel at a site distal of the stenosis before the angioplasty or stenting procedure takes place. In a deployed configuration, the embolic protection device is intended to act as a filter that allows blood to pass but traps embolic particles traveling downstream.

For example, an embolic protection device may be attached to a wire guide and encased within a sheath, and then loaded into a guiding catheter for delivery to a site proximal of the stenosis. A clinician may advance the embolic protection device and the sheath surrounding it out of the distal end of the guiding catheter and across the stenosed region by pushing on the wire guide. Once the device is positioned at a site distal of the stenosis, the clinician may remove (e.g., retract) the sheath to deploy the embolic protection device to an expanded configuration for use. The embolic particles trapped in the expanded device may be removed from the vessel by collapsing the device and retracting the wire guide.

In about 20-30% of carotid stenting situations, however, the anatomy of the vessel may be too tortuous to permit placement of the embolic protection device in the above-described manner using existing delivery systems. A tortuous vessel may contain severe bends, kinks or coils that can inhibit delivery of the embolic protection device. In view of this problem, the inventor believes a new embolic protection device delivery system capable of navigating a tortuous vasculature would be desirable.

### BRIEF SUMMARY

A delivery system for an embolic protection device is described herein. The delivery system may allow the embolic protection device to be transported and deployed within vessels having a tortuous anatomy.

The delivery system for the embolic protection device includes a delivery catheter having a bend near a distal end thereof. The bend has a non-included angle of greater than about 40 degrees relative to a longitudinal axis of the catheter. The delivery system further comprises an embolic protection device disposed within the catheter.

### BRIEF DESCRIPTION OF THE DRAWINGS

FIG. 1 is a schematic of the human body showing intraluminal access to the left common carotid artery through the femoral artery;

FIG. 2 is a schematic of the aortic arch and a stenosed region in the left common carotid artery;

FIG. 3 is a cross-sectional schematic of a distal portion of an intraluminal delivery system including a delivery catheter and an embolic protection device contained therein, according to one embodiment;

FIGS. 4A-4C are a cross-sectional views of an intermediate region between the distal and proximal portions of the delivery system, according to several embodiments.

FIGS. 5A-5E are cross-sectional views of a control wire of the delivery system, according to several embodiments.

FIG. 6 schematically shows the positioning of the delivery system within a vessel in preparation for delivery and deployment of the embolic protection device;

FIG. 7 schematically shows the embolic protection device being advanced across the treatment site; and

FIG. 8 schematically shows an embolic protection filter in a deployed configuration.

### DETAILED DESCRIPTION

Described herein is an intraluminal delivery system 5 that may be suitable for directing and delivering an embolic protection device 10 to a site distal of a stenosis in a tortuous vessel. The delivery system 5 may advantageously allow carotid stenting procedures to be carried out under a wider range of circumstances.

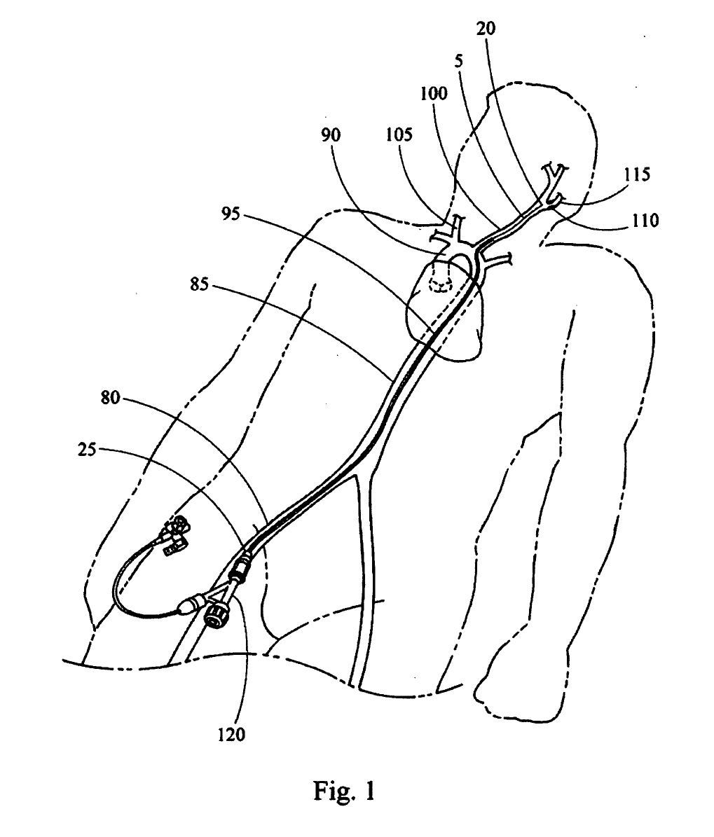

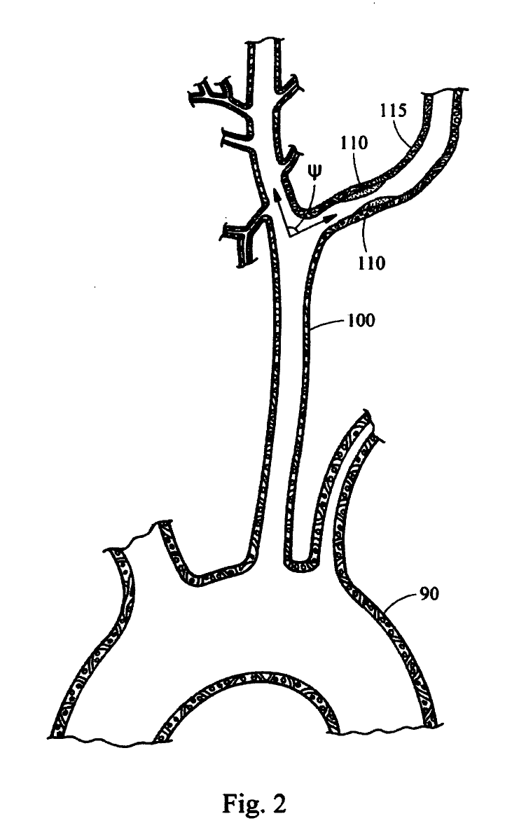

For example, the intraluminal delivery system 5 may be used to deliver an embolic protection device 10 to a treatment site (e.g., a stenosed region) in a tortuous carotid artery. Typically, to access a carotid artery, a percutaneous incision is made in the femoral artery 80 and an intraluminal medical device is advanced through the aorta 85 and the aortic arch 90. This access route is shown schematically in FIG. 1. Branching from the aortic arch 90 are the left common carotid artery 100 and the right common carotid artery 105, which supply blood to the head and neck, as shown in FIG. 2. A stenosed region 110 is shown in the internal carotid artery 115 extending from the left common carotid artery 100 at a take-off angle of ψ. A guiding sheath 95 may be placed within the carotid artery of interest prior to insertion and delivery of the intraluminal medical device, as shown in FIG. 1 and as will be further discussed below. Alternatively, other vascular pathways besides the femoral artery, such as, for example, a radial or brachial artery, may be employed to access the carotid artery of interest.

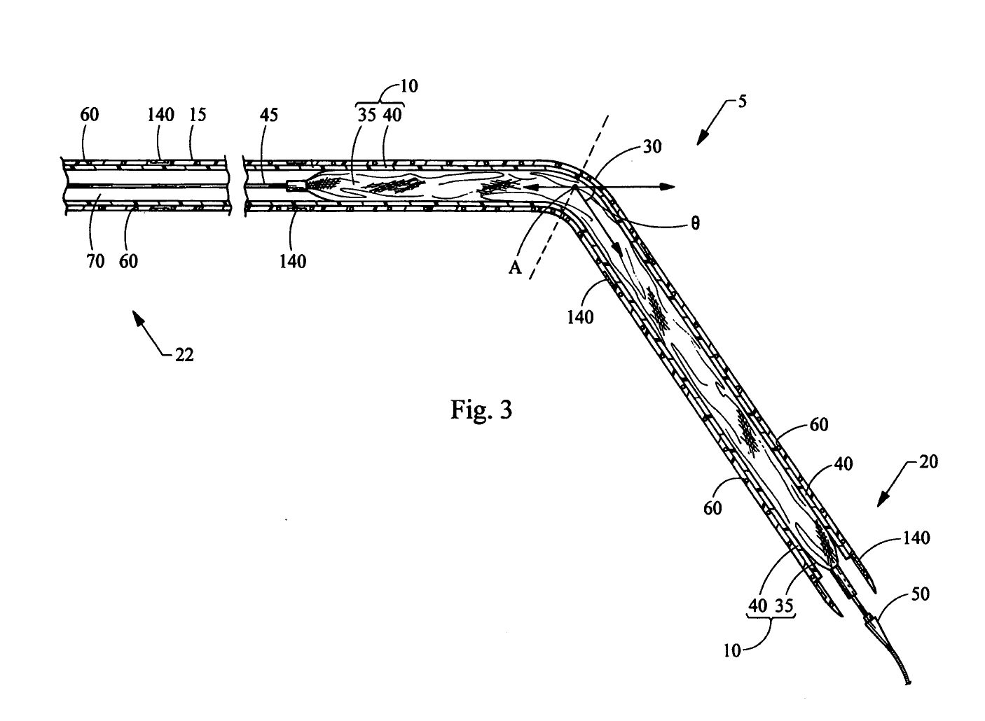

As shown schematically in FIG. 3, the intraluminal delivery system 5 of the present disclosure includes a delivery catheter 15 having a distal end 20, a proximal end 25, and a bend 30 near the distal end 20. The bend 30 is defined by a non-included angle θ of at least about 40 degrees relative to a longitudinal axis of the catheter 15. The delivery system 5 also includes an embolic protection device 10 disposed within the delivery catheter 15 in a collapsed or undeployed configuration.

According to one embodiment, the embolic protection device 10 may include an embolic protection filter 35 disposed within a delivery sheath 40. Suitable filters may include, for example, Angioguard™ RX, a product of Cordis (Miami Lakes, Fla.); RX Accunet™, a product of Guidant (Indianapolis, Ind.); FilterWire EZ, a product of Boston Scientific (Natick, Mass.); and EmboShield, a product of Abbott Vascular Devices (Redwood City, Calif.). Depending on the filter design, a wire guide 45 may be attached to the filter 35 at the proximal end thereof, or the filter 35 may pass over the wire guide 45 during delivery of the device. Of the representative filters 35 mentioned above, the EmboShield device is an “over-the-wire” device, while the others are “fixed-wire” filters. The exemplary embolic protection filter 35 shown in FIG. 3 is a fixed wire filter.

The bend 30 may be disposed in the range of from about 0.5 cm to about 2.0 cm from the distal end 20. Preferably, the bend 30 is disposed in the range of from about 1.0 cm to about 1.5 cm from the distal end 20. For the purposes of the present disclosure, the distance of the bend 30 from the distal end 20 may be approximately determined by bisecting the bend with a line to determine point A, as shown in FIG. 3, and then measuring the distance from point A to the distal end 20.

The embolic protection device 10 may be disposed at the distal end 20 of the delivery catheter 15. The delivery catheter 15 and the sheath 40 may be substantially concentric and define a lumen 70 through which the filter 35 and wire guide 45 may pass. The distal tip or nose cone 50 of the embolic protection device 10 may protrude from the distal end 20 of the delivery catheter 15, while the rest of the embolic protection device 10 is preferably disposed therein. Due to the length of the device 10, a portion of the embolic protection device 10 may be positioned along the bend 30. A typical embolic protection device 10 is about 2 cm to 3 cm in length (not including a wire guide 45 that may be attached to the proximal end of the device 10), although other lengths are possible. Preferably, the embolic protection device 10 is sufficiently flexible for placement along the bend 30 of the delivery catheter 15 and also for navigating tortuous anatomy.

The non-included angle θ is preferably at least about 40 degrees. Non-included angles θ of about 40 degrees and larger may prove advantageous in accessing and traversing tortuous vessels. In use, the catheter may be rotated and maneuvered within the vessel to direct the distal end through a tortuous region or into a branch artery having a significant angle of take off from the main stem, as shown for example in FIG. 3. According to one embodiment, the non-included angle θ may be in the range of from about 50 degrees to about 80 degrees. Preferably, the non-included angle θ may be in the range of from about 60 degrees to about 70 degrees.

The delivery catheter 15 may have a low profile that enables the device to reach and cross a narrow restriction, such as a tight stenosis, in a small diameter vessel. For example, the catheter 15 may be about 6.0 French or less in size (outer diameter). The catheter 15 may have an inner diameter sized to contain an embolic protection device 10 of about 4.0 French or less in size. Preferably, the delivery catheter 15 may be sized to contain an embolic protection device 10 of about 3.7 French or less in size.

The delivery catheter 15 may include one or more radiopaque markers 140 near the distal end. The radiopaque markers 140 may be thin-walled tubular structures formed from radiopaque materials, such as, for example, gold, tungsten, platinum, palladium, or alloys thereof. The radiopaque markers 140 may be secured about the circumference of the delivery catheter 15 to improve the visibility of the catheter 15 during noninvasive imaging procedures, such as x-ray fluoroscopy.

According to one embodiment, the delivery catheter 15 may include at least two radiopaque markers 140. For example, three, four, five, six or more radiopaque markers 140 may be used. The markers 140 may be spaced at a predetermined distance along the catheter 15 and used for calibrating distances during imaging procedures. For example, five markers 140 spaced along the delivery catheter 15 a distance of 1 cm apart, as shown in FIG. 3, may be used to calibrate a 4 cm distance in an image.

The radiopaque markers 140 may be secured to the delivery catheter 15 by, for example, applying an axial tensile force to the catheter 15 to cause a tensile expansion and a radial contraction thereof, and then sliding the one or more markers 140 over the catheter 15 before releasing the force. Upon release of the force, the catheter 15 may radially expand, and the radiopaque markers 140 may be secured about the circumference of the catheter 15.

Preferably, the delivery catheter 15 may be sufficiently “pushable” such that longitudinal forces can be effectively transmitted along the length of the catheter 15 from its proximal end 25, where external manipulations by the clinician take place, to its distal end 20, which is disposed inside the vasculature. It is also desirable that the delivery catheter 15 be sufficiently “torqueable” that rotational forces may be effectively transmitted from the proximal end 25 to the distal end 20. The catheter 15 may therefore include a reinforcement structure 60 made of a biocompatible metal or alloy, such as stainless steel, for increased longitudinal stiffness. Such a reinforcement structure 60 may be embedded within the catheter wall and take the form of a braid, mesh, coil or other arrangement, as shown in FIG. 3. A description of such reinforcement structures 60, as well as methods of manufacturing medical devices including such reinforcement structures 60, may be found in U.S. Pat. Nos. 5,700,253 and 5,380,304, the contents of which are incorporated by reference herein.

The embedded reinforcement structure 60 may be circumferentially disposed within the wall of the catheter 15 and may extend along at least a portion of the length thereof. According to one embodiment, the reinforcement structure 60 may be a round wire or flat wire in a helical configuration (e.g., a coil) that extends from a position proximal of a tip region of the catheter 15 to a position at or near the proximal end of the catheter. Alternatively, the reinforcement structure 60 may include multiple wires formed into a braid or mesh. The reinforcement structure 60 may extend along substantially the entire length of the catheter 15, according to one embodiment.

According to another embodiment, the reinforcement structure 60 may be a wire adapted to rotate and/or flex the catheter 15 in the vicinity of the bend 30. The wire may extend from the proximal end 25 through at least a portion of the length of the catheter 15. The wire may be disposed in the wall of the catheter 15 or in a lumen (e.g., 70). According to this embodiment, the angle and orientation of the bend 30 may be changed by external manipulation of the wire at the proximal end 25 of the catheter 15. For example, a wire initially extending to a position proximal of the bend 30 may be moved in a distal direction to pass through and consequently straighten the bend 30. A wire as described in U.S. Pat. No. 5,820,592, which is hereby incorporated by reference, may also be used. For example, the wire may extend longitudinally through the catheter wall and have a connection with a proximal actuator. Manipulation of the actuator may rotate the wire, for example, and allow the orientation of the distal end of the catheter to be controlled.

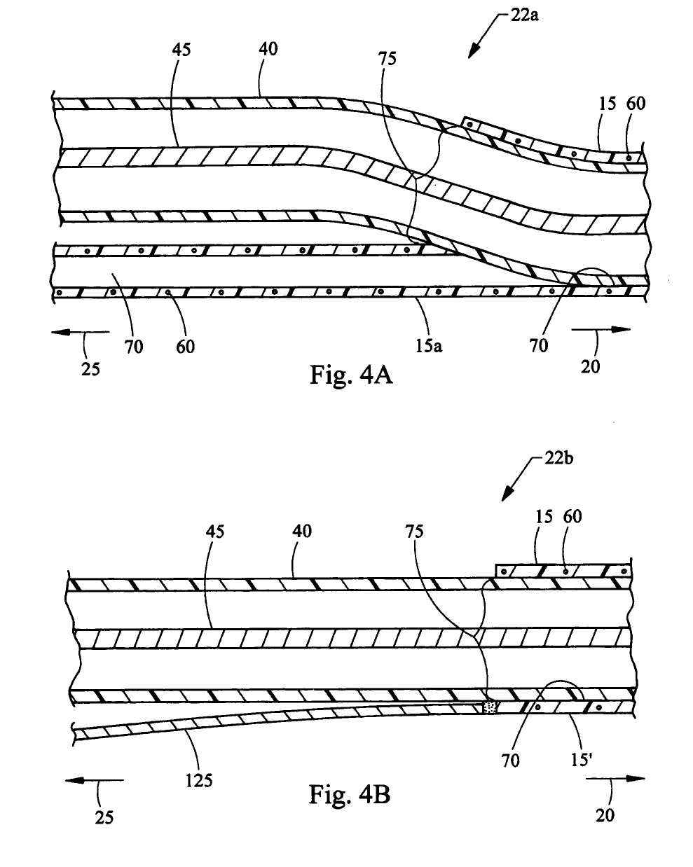

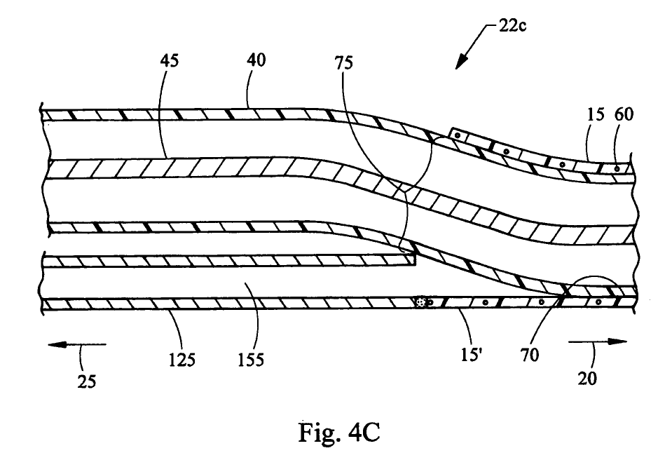

According to one embodiment, the delivery catheter 15 may have an “over-the-wire” configuration in which the sheath 40 and the wire guide 45 exit the lumen 70 at the proximal end 25. According to an alternative embodiment, the delivery catheter 15 may have a rapid exchange design in which the sheath 40 and the wire guide 45 exit the lumen 70 at an exit port 75 in an intermediate region 22 between the distal and proximal ends 20, 25. Exemplary embodiments of a rapid exchange design of the delivery catheter 15 are shown in FIGS. 4A-4D. The exit port 75 may be disposed from about 8 cm to about 25 cm from the distal end 20. Alternatively, the exit port 75 may be disposed from about 12 cm to about 18 cm from the distal end. In one particular embodiment, the exit port may be disposed about 15 cm from the distal end. The rapid exchange (or “monorail”) design may simplify the process of removing the catheter 15 from a vessel in a patient's body after the embolic protection filter 35 of the present disclosure has been deployed, as will be further described below.

FIG. 4A illustrates one embodiment of the rapid exchange design of the delivery system 5. Referring to the figure, which shows a cross-sectional view of the intermediate region 22a between the distal and proximal ends 20, 25 of the delivery system 5, the exit port 75 may be disposed in the wall of the delivery catheter 15. The sheath 40 and wire guide 45 (which may be attached to the filter 35 shown in FIG. 3) may pass through the exit port 75. The delivery catheter 15 may have a portion 15a of reduced diameter proximal of the exit port 75 to minimize the combined profile of the sheath 40 and the catheter 15. Preferably, the lumen 70 in the portion 15a of reduced diameter is large enough to deliver contrast fluid to a body vessel.

FIG. 4B illustrates another embodiment of the rapid exchange design of the delivery system 5. The figure shows a cross-sectional view of the intermediate region 22b between the distal and proximal ends 20, 25 of the delivery system 5. The sheath 40 and wire guide 45 (which may be attached to the filter 35 shown in FIG. 3) may exit the lumen 70 of the delivery catheter 15 through the exit port 75. According to this embodiment, the exit port 75 corresponds to the proximal end 15′ of the delivery catheter 15. Accordingly, the delivery catheter 15 has a length that does not extend all the way from the distal end 20 to the proximal end 25 of the delivery system 5. The delivery catheter 15 may have a length of, for example, between about 8 cm and about 25 cm, according to this embodiment. A control wire 125 may be attached to the proximal end 15′ of the catheter 15. The control wire 125 extends in a proximal direction from the proximal end 15′ of the delivery catheter 15 to the proximal end 25 of the delivery system 5 to allow for external control over the positioning of the catheter 15.

FIG. 4C illustrates another embodiment of the rapid exchange design of the delivery system 5. According to this embodiment, the control wire 125 attached to the proximal end 15′ of the catheter 15 is hollow. That is, the control wire 125 includes a lumen 155. FIG. 4C shows a cross-sectional view of the intermediate region 22c between the distal and proximal ends 20, 25 of the delivery system 5. The sheath 40 and wire guide 45 (which may be attached to the filter 35 shown in FIG. 3) may exit the lumen 70 of the delivery catheter 15 through the exit port 75. The exit port 75 corresponds to the proximal end 15′ of the delivery catheter 15. Accordingly, the delivery catheter 15 has a length that does not extend all the way from the distal end 20 to the proximal end 25 of the delivery system 5. The delivery catheter 15 may have a length of, for example, between about 8 cm and about 25 cm, according to this embodiment. The control wire 125 extends in a proximal direction from the proximal end 15′ of the delivery catheter 15 to the proximal end 25 of the delivery system 5 to allow for external control over the positioning of the catheter 15.

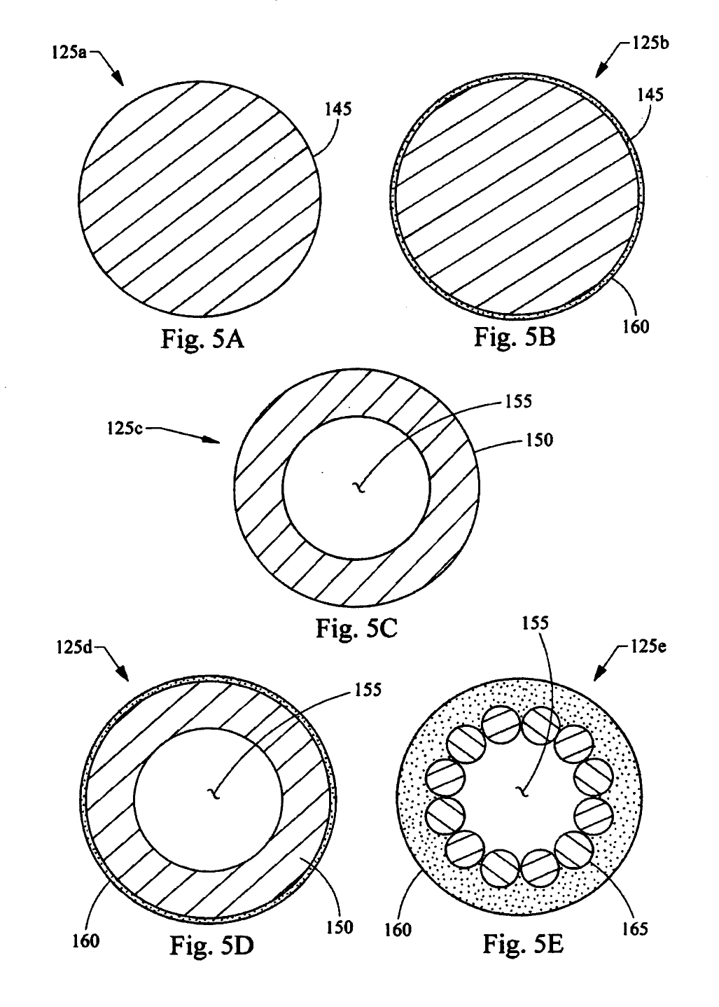

Referring to the cross-sectional schematics of the control wire 125 shown in FIGS. 5A and 5B, the control wire 125 may be a solid wire. Alternatively, as shown in FIGS. 5C, 5D, and 5E, the control wire 125 may have a lumen 155. The control wire 125 may be a round wire with a circular cross-section, as shown in FIGS. 5A-5E. However, other cross-sectional shapes are also possible. For example, the control wire 125 may be a flat wire with a rectangular cross-section.

The control wire 125 may include a polymeric coating 160, as shown in FIGS. 5B, 5D, and 5E. The polymeric coating 160 may aid in securing the control wire 125 to the delivery catheter 15. For example, the presence of a polymeric coating 160 may permit the control wire to be bonded to the catheter 15. Also, the polymeric coating 160 may improve the lubricity or other properties of the control wire 125.

According to one embodiment, as shown in FIG. 5E, the control wire 125 may have a coaxial structure including a plurality of wires 165. The control wire of this embodiment may also include a lumen 155. A polymeric coating 160 may further be disposed on an outer surface of the plurality of wires 165.

The control wire 125 may have a length of about 20 cm or longer and a diameter in the range of from about 0.1 mm to about 0.5 mm. If the control wire 125 includes a lumen 155, then the control wire 125 may have an outer diameter in the range of from about 0.2 mm to about 0.6 mm and an inner diameter in the range of from about 0.1 mm to about 0.4 mm. Preferably, the lumen of the control wire 125 may be large enough to deliver contrast fluid to the body vessel.

To secure the control wire 125 to the proximal end 15′ of the delivery catheter 15, the control wire 125 may be adhesively or thermally bonded to the proximal end 15′, as shown, for example, in FIGS. 4B and 4C. Alternatively, a portion of the control wire 125 may be embedded in the wall of the delivery catheter 15. For example, the portion of the control wire 125 may extend into the wall a distance of about 0.5 cm or less from the proximal end 15′. In another example, the portion may extend into the wall a distance of about 5.0 cm or less from the proximal end 15′. According to some embodiments, the portion may extend into the wall substantially all the way from the proximal end 15′ to the distal end of the delivery catheter 15. Within the wall, the portion of the control wire 125 may have a substantially linear configuration and/or may be disposed about the circumference of the delivery catheter 15. For example, the portion may take the form of a helical coil within the wall of the catheter 15 that wraps around the circumference and extends along at least a portion of the length. Processing methods known in the art and described above for embedding the reinforcement structure 60 in the wall of the catheter 15 may be applied to embed the portion of the control wire 125 within the wall of the catheter 15.

A method for delivering an embolic protection device to a site distal of a stenosis in a tortuous vasculature is described.

First, a clinician may perform a carotid angiography procedure to obtain a map of the vasculature. The procedure may entail inserting a flush catheter into the common carotid artery 100 and injecting a contrast fluid or dye which is visible under x-ray irradiation. The resulting pictures, called angiograms, allow the clinician to visualize the area and measure the take-off angles of the arteries of interest. Depending on the geometry and configuration of the vessels, use of the delivery catheter 15 may be advantageous. If, for example, the take-off angle ψ of the internal carotid artery 115 which contains a stenosed region 110 is greater than about 45 degrees with respect to the common carotid artery 100, as shown in FIG. 2, use of the delivery catheter 15 may be advisable.

Next, the embolic protection device 10 to be used in the procedure may be prepared according to the manufacturer's instructions and then front-loaded or back-loaded into the delivery catheter 15. Depending on the filter design, a wire guide 45 may be attached to the filter 35 at the proximal end, or the filter 35 may pass over the wire guide 45 during delivery of the device, as discussed above. During front-loading of the embolic protection device 10 into the delivery catheter 15, the collapsed filter 35 contained within the sheath 40 may be advanced within the delivery catheter 15 in a distal direction. For example, the delivery catheter 15 may be flushed with saline and the embolic protection device 10 may be advanced through the catheter 15 from the proximal end 25 to the distal end 20. In the case of a monorail or rapid exchange catheter design, the embolic protection device 10 may be loaded into the delivery catheter 15 through the exit port 75a positioned between the distal and the proximal ends 20, 25. Alternatively, the embolic protection device 10 may be back-loaded into the delivery catheter 15. In this case, the device 10 may be loaded into the delivery catheter 15 in a proximal direction through the distal end 20.

In the loaded configuration, a portion of the embolic protection device 10 may protrude from the distal end 20 of the delivery catheter 15. For example, the distal tip or nose cone 50 of the embolic protection device 10 may protrude from the distal end 20 with the rest of the embolic protection device 10 preferably disposed within the delivery catheter 15. Alternatively, the embolic protection device 10 may be contained entirely within the delivery catheter 15 for delivery to the treatment site.

Prior to delivery of the embolic protection device 10 to the treatment site using the delivery catheter 15, a guiding sheath or guiding catheter 95 may be placed into the common carotid artery 100 for use as a channel to access the stenosed region. The protocol described in a publication by Peter A. Schneider of Hawaii Permanente Medical Group (Peter A. Schneider, Access for Carotid Interventions, in CAROTID INTERVENTIONS, 93-109 (Peter A. Schneider et al. eds., 2004)) may be used, for example. (This publication is hereby incorporated by reference.) Alternatively, other sheath or catheter placement techniques known in the art may be employed. Preferably, the distal end of the sheath or catheter 95 is placed far enough into the common carotid artery 100 to avoid recoiling back into the arch 90. On the other hand, the sheath or catheter 95 is preferably not placed so far into the artery 100 that the branch containing the lesion 110 is obstructed. An exemplary guiding sheath 95 which may be employed in this procedure is the Shuttle Select™ Guiding Sheath of Cook Medical, Inc. (Bloomington, Ind.)).

Once the guiding sheath or guiding catheter 95 has been placed in the common carotid artery 100, the delivery catheter 15, including the embolic protection device 10, may be advanced through the arch 90 and the guiding sheath/catheter 95 to a site in the common carotid artery 100 proximal of the stenosis 110. The process may be guided by fluoroscopy, that is, the x-ray tracking of one or more radiopaque markers attached to the delivery catheter 15 and/or the embolic protection device 10.

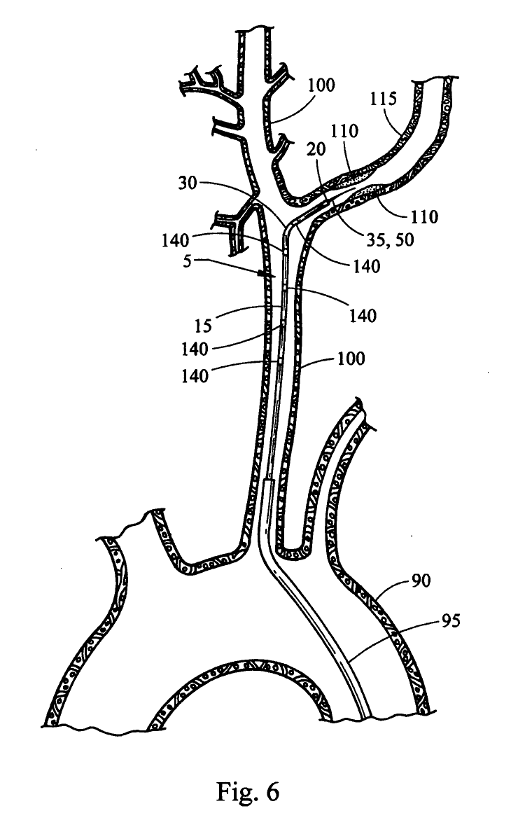

Once the embolic protection device delivery system 5 has been placed in the common carotid artery 100, the bend 30 of the delivery catheter 15 may be exploited to generally align the distal end 20 of the catheter 15 in the direction of the artery 115 that contains the stenosis 110. For example, guided by fluoroscopy, the delivery catheter 15 may be rotated and maneuvered such that the distal end 20 is positioned in alignment with the left internal carotid artery 115, as shown schematically in FIG. 6. This ability to aim the distal end 20 of the embolic protection device delivery system 5 in this fashion may be particularly advantageous in accessing and traversing tortuous vessels.

Depending on the location of the stenosed region 110 within the artery 115, the embolic protection device 10 may at this point be ejected from the distal end 20 of the delivery catheter 15 in the direction of the treatment site 110 in preparation for deployment. Alternatively, the delivery catheter 15 itself may be directed into the artery 115 to obtain closer access to the treatment site 110 before advancing the embolic protection device 10 out of the distal end 20. In the latter case, the bend 30 of the delivery catheter 15 may be further exploited to direct the distal end 20 of the delivery system 5 in the desired direction when additional tortuosity is encountered within the vessel. The reinforcement structure 60 disposed within the catheter wall according to some embodiments may further aid in traversing tortuous regions by increasing the pushability and/or torqueability of the delivery catheter 15. Preferably, the distal end 20 of the catheter 15 is positioned proximal of the stenosed region 110 and does not cross the stenosed region 110.

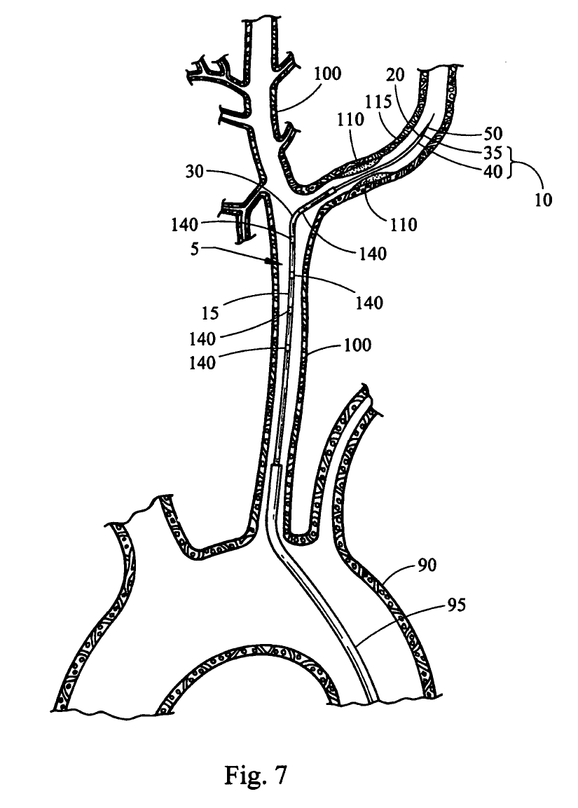

The embolic protection device 10 may be ejected from the distal end 20 of the delivery catheter 15 by pushing on the wire guide 45 if the embolic protection device 10 is a fixed-wire device with a wire guide 45 attached to the proximal end. Alternatively, in the case of an over-the-wire device, the embolic protection device 10 may be ejected from the delivery catheter 15 by pushing on the proximal end of the device 10 using a push rod or similar component that may be threaded over the wire guide 45. In both cases it may be desirable to keep the delivery catheter 15 substantially stationary when the embolic protection device 10 is being ejected from the distal end 20 of the catheter 15 and across the stenosed region 110, as shown in FIG. 7.

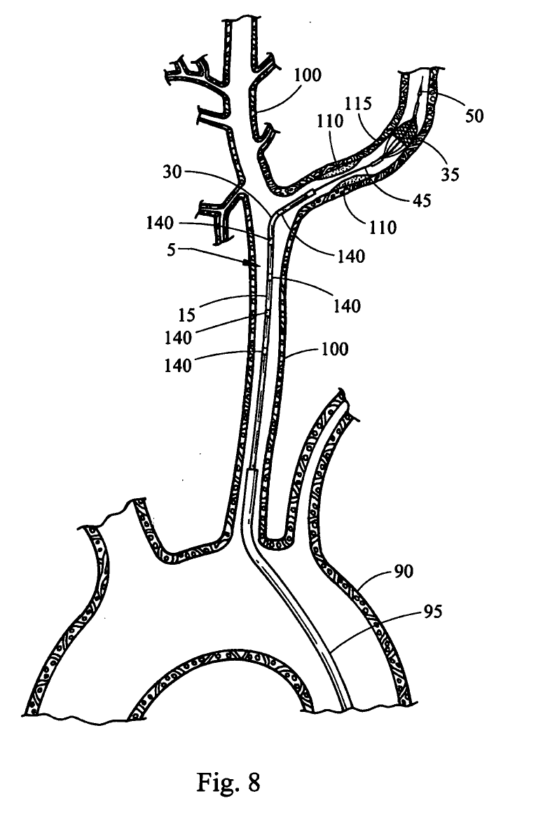

The embolic protection device 10 may be deployed at a site distal of the stenosed region 110 by removing the delivery sheath 40. Depending on the filter design, removal of the delivery sheath 40 may entail retracting the sheath 40 in a proximal direction, or peeling away the sheath 40. Upon removal of the sheath 40, the filter 35 may expand to a deployed configuration within the artery 115. In the deployed configuration, which is shown in FIG. 8, the filter 35 may be able to trap embolic particles generated during an angioplasty procedure and/or deployment of an expandable stent at the stenosed region 110.

The delivery catheter 15 may be removed from the patient's body after delivery and/or deployment of the embolic protection device 10. The rapid exchange (or “monorail”) design described above may simplify the process of removing or retracting the catheter 15. Preferably, the distal positioning of the wire guide and filter remain substantially unchanged during the retraction. To maintain the internal positioning of the wire guide and filter as the catheter is retracted, a length of wire guide corresponding to the length within the catheter lumen preferably extends outside the patient's body. Consequently, when a catheter overlies a wire guide over its entire length in an over-the-wire configuration, a substantial length of wire guide extends outside of the patient's body. In contrast, in the case of a catheter having a rapid exchange design, the wire guide exits the lumen at an exit port in an intermediate region between the distal and proximal ends, and thus a substantially shorter length of the catheter overlies the wire guide. A shorter length of wire guide may extend outside the patient's body, and the retraction process may be considerably simplified. A single clinician may be able to exchange out the catheter without assistance, whereas a medical assistant may be needed during retraction of an over-the-wire catheter.

If desired, the delivery catheter 15 may later be reinserted into the artery 115 to collapse and retrieve the embolic protection filter 35 after completion of the procedure. This may be particularly advantageous due to the torqueability of the delivery catheter 15. In some cases, as a recovery catheter is being retracted with the collapsed filter inside, the recovery catheter, which is relatively soft, may become trapped by the open cells of the deployed stent. In such a situation, the torqueability of the present delivery catheter 15 may prove advantageous to free the catheter 15 from the stent and remove the collapsed embolic protection filter 35 from the patient.

The delivery catheter 15 may be made of one or more polymers, such as, for example, a polyamide (e.g., nylon), fluorocarbon (e.g., polytetrafluoroethylene (PTFE)), polyether block amide (PEBA), polyolefin, or polyimide. As previously described, the catheter may further include a metallic (e.g., stainless steel) reinforcement structure 60 embedded within the one or more polymers to impart kink resistance and column strength to the catheter. Conventional catheter manufacturing methods known in the art, including, for example, extrusion and/or molding, may be employed to fabricate the catheter 15. In particular, the bend 30 in the delivery catheter 15 may be formed by molding techniques.

A delivery system 5 for an embolic protection device 10 has been described herein. The delivery system 5 may allow the embolic protection device 10 to be transported and deployed within vessels having a tortuous anatomy, making it possible to carry out carotid stenting procedures under a wider range of circumstances.

Although the present invention has been described with reference to certain embodiments thereof, other embodiments are possible without departing from the present invention. The spirit and scope of the appended claims should not be limited, therefore, to the description of the preferred embodiments contained herein. All embodiments that come within the meaning of the claims, either literally or by equivalence, are intended to be embraced therein. Furthermore, the advantages described above are not necessarily the only advantages of the invention, and it is not necessarily expected that all of the described advantages will be achieved with every embodiment of the invention.

1. A delivery system for an embolic protection device, comprising:
a catheter comprising a bend near a distal end thereof, the bend having a non-included angle of greater than about 40 degrees relative to a longitudinal axis of the catheter; and
an embolic protection device disposed within the catheter.

2. The delivery system according to claim 1, wherein the embolic protection device includes an embolic protection filter disposed within a delivery sheath, at least a portion of the delivery sheath being disposed within the catheter.

3. The delivery system according to claim 1, wherein the bend is disposed in the range of between about 0.5 cm and about 2.0 cm from the distal end.

4. The delivery system according to claim 3, wherein the bend is disposed in the range of between about 1.0 cm and about 1.5 cm from the distal end.

5. The delivery system according to claim 1, wherein the non-included angle is in the range of between about 50 degrees and about 80 degrees.

6. The delivery system according to claim 5, wherein the non-included angle is in the range of between about 60 degrees and about 70 degrees.

7. The delivery system according to claim 1, wherein the catheter is sized to contain an embolic protection device of about 4.0 French or less in size.

8. The delivery system according to claim 1, wherein a portion of the embolic protection device is disposed along the bend.

9. The delivery system according to claim 1, wherein the catheter further comprises a reinforcement structure disposed in a wall thereof and extending along at least a portion of a length thereof.

10. The delivery system according to claim 9, wherein the reinforcement structure comprises a wire in a helical configuration.

11. The delivery system according to claim 9, wherein the reinforcement structure comprises a wire adapted to vary the non-included angle of the bend.

12. The delivery system according to claim 1, wherein the catheter further comprises an exit port disposed a distance of from about 8 cm to about 25 cm from the distal end, and wherein a second diameter of the catheter proximal of the exit port is smaller than a first diameter of the catheter distal of the exit port.

13. The delivery system according to claim 1, wherein the catheter has a length of from about 8 cm to about 25 cm, and wherein a control wire extending in a proximal direction is secured to the catheter.

14. The delivery system according to claim 13, wherein the control wire includes a wire lumen in communication with a lumen of the catheter.

15. The delivery system according to claim 13, wherein the control wire comprises a multiplicity of wire strands.

16. The delivery system according to claim 13, wherein the control wire comprises a polymeric coating.

17. The delivery system according to claim 1, wherein the catheter includes at least two radiopaque markers disposed along a length thereof with a predetermined spacing therebetween.

18. The delivery system according to claim 1, wherein the bend is disposed in the range of between about 0.5 cm and about 2.0 cm from the distal end, and wherein the non-included angle is in the range of between about 50 degrees and about 80 degrees,
wherein the embolic protection device includes an embolic protection filter disposed within a delivery sheath, at least a portion of the delivery sheath being disposed within the catheter, and wherein a portion of the embolic protection device is disposed along the bend,
wherein the catheter further comprises a reinforcement structure disposed in a wall thereof and extending along at least a portion of a length thereof, the reinforcement structure comprising a wire in a helical configuration,
wherein the catheter is sized to contain an embolic protection device of about 4.0 French or less in size, and
wherein the catheter includes at least two radiopaque markers disposed along a length thereof with a predetermined spacing therebetween.

19. The delivery system according to claim 18, wherein the catheter further comprises an exit port disposed a distance of from about 8 cm to about 25 cm from the distal end, and wherein a second diameter of the catheter proximal of the exit port is smaller than a first diameter of the catheter distal of the exit port.

20. The delivery system according to claim 18, wherein the catheter has a length of from about 8 cm to about 25 cm, and wherein a control wire extending in a proximal direction is secured to the catheter.
